(19) United States
(12) Patent Application Publication
(10) Pub. No.: US 2016/0310512 A1
(43) Pub. Date: Oct. 27, 2016

(54) THERAPY REGIMEN FOR TREATING SEVERE ACNE RELATED DISEASES

(71) Applicant: GALDERMA RESEARCH & DEVELOPMENT, Biot (FR)

(72) Inventors: Fabien AUDIBERT, La Roquette Sur Siagne (FR); Nabil KERROUCHE, Le Rouret (FR); Pascale SOTO, Antibes (FR)

(21) Appl. No.: 15/105,813

(22) PCT Filed: Dec. 18, 2014
(86) PCT No.: PCT/EP2014/078510

§ 371 Date: Jun. 17, 2016

Related U.S. Application Data
(60) Provisional application No. 61/918,391, filed on Dec. 19, 2013.

(51) Int. Cl. A61K 31/65; A61K 9/48; A61K 31/327; A61K 9/06; A61K 9/00; A61K 31/192

(52) U.S. Cl. CPC A61K 31/65; A61K 9/0014; A61K 9/0053; A61K 31/192; A61K 31/327; A61K 9/06; A61K 9/48

(57) ABSTRACT

A therapy regimen is described for the treatment of severe acne related diseases preferentially severe or nodular acne. The therapy regimen adds a topical fixed-dose combination of a retinoid and an anti-bacterial agent, such as benzoyl peroxide, to a course of oral antibiotic therapy using a daily dose of antibiotic ranging from 150 mg to 300 mg per day.

Also described, is a fixed-dose combination of a retinoid and an anti-bacterial agent for use in the therapy regimen, and a kit suitable for implementing such therapy.

### FIELD OF THE INVENTION

This invention relates to a new therapy regimen for treating severe acne related diseases, particularly severe acne vulgaris. The regimen includes a topical treatment with a fixed-dose combination of a retinoid, such as Adapalene, and an anti-bacterial agent, such as benzoyl peroxide (BPO), and an oral antibiotic drug.

### BACKGROUND OF THE INVENTION

The burden of acne is significant. More than 50 million Americans experience some form of acne. Acne vulgaris is a common skin disorder that makes up 20% of the visits to dermatologists, and affects approximately 80% of young adults and adolescents. Management of acne is challenging, especially considering the chronicity of the disease and the variability in response to treatments. Acne management can be complex, because the disease is multifactorial, involving various etiological features, including follicular hyperkeratinisation, increased sebum production, P. acnes proliferation, and inflammation.

Oral isotretinoin (13-cis-retinoic acid) is currently the only medication that affects all of the major acne pathogenic factors. However, this drug has been associated with multiple serious side effects, the most serious of which is teratogenicity. Therefore, for inflammatory acne, except for the most severe or aggressive cases of the disease, alternative treatments, such as the combination of an oral antibiotic and a topical treatment, should be the preferred option.

The bulk of the current evidence for topical retinoid—oral antibiotic combination therapy in inflammatory acne is with Adapalene 0.1%. One study in particular demonstrated that the combination of 100 mg of Doxycycline with Adapalene gel 0.1% led to a greater and faster improvement compared to the oral antibiotic alone. See “Combination therapy with adapalene gel 0.1% and doxycycline for severe acne vulgaris: a multicenter, investigator-blind, randomized, controlled study”, Skinmed. 2005, May-June; 4(3):138-46.

The recent Consensus Recommendations for the Management of Acne (JAAD sup 2003; 49:1) states that effective acne treatment should target as many of its pathogenic factors as possible.

The recommendations also state that a topical retinoid should be used in the initial treatment of almost all new patients with acne, because they are the most effective anticomedonal agents currently available. Retinoids help disrupt acne pathogenesis by preventing the development of new microcomedones, and some possess both direct and indirect anti-inflammatory activity.

The management of acne often requires combination therapy and a long-term therapeutic strategy. See, for example, Thiboutot D. New treatments and therapeutic strategies for acne, Arch Family Med 2000; 9: 179-187; Gollnick H., et al. Management of Acne, a report from a Global Alliance to Improve Outcomes in Acne, J. Am. Acad. Dermatol., 2003; 49(1 suppl):S1-537).

Dreno et al. in Eur J Dermatol 2004; 14: 391-9, present a set of recommendations on the use of oral antibiotics in acne, developed by a group of European acne specialists, designed to be considered by dermatologists and general practitioners to cover optimal choice of antibiotic, drug doses, duration of treatment, combination treatment, and maintenance therapy.

Marissa D. Newman et al. in Am J Clin Dermatol 2011; 12 (1) a reviewed treatment modalities for severe nodular acne; and provides novel ways of treatment in order to target the multifactorial pathogenesis of the disease.

Recently, a unique fixed-dose combination of adapalene and benzoyl peroxide in a form of gel (Adapalene BPO Gel) has been granted with Marketing Authorization in Europe and US under the trade name of Epiduo® (Galderma). Adapalene BPO Gel is a unique antibiotic-free combination of Adapalene 0.1%, a well-tolerated and efficacious topical retinoid, and BPO 2.5%, a well established antimicrobial agent. The complementary modes of action, efficacy and safety profiles of these two agents make Adapalene BPO Gel the most appropriate choice for once-daily treatment for all types of acne except for the most severe cases. Adapalene (6-[3-(1-adamantyl)-4-methoxyphenyl]-2-naphtoic acid) possesses anticomedogenic, comedolytic, and anti-inflammatory properties whereas BPO, the most potent bactericidal agent, is more effective than other topical antibiotics against P. acnes. See “Adapalene-Benzoyl Peroxide, A Unique Fixed-Dose Combination Gel For Acne treatment: A Randomized, Double-Blind, Controlled Trial In 1668 Patients” (http://www.galdermanordic.com/sverige/pdf/FP0039.pdf). Because neither retinoids nor BPO creates selective pressure for resistance, this combination may be expected to decrease the incidence of epidermal bacterial resistance relative to antibiotics. Furthermore, unlike tretinoin, Adapalene is stable in the presence of light when combined with BPO. See “Adapalene-Benzoyl Peroxide Combination Effective and Safe for Acne”, CME Released: 11/09/2007; Valid for credit through Nov. 9, 2008.

Efficacy and safety of Adapalene BPO Gel has been established in a large clinical program. Adapalene BPO Gel combination provides significantly greater efficacy for the treatment of moderate acne vulgaris and a quicker onset of action relative to respective monotherapies, with a comparable safety and tolerability profile relative to Adapalene.

Moreover, acne vulgaris is a multi-factorial disease characterized by:
Overproduction of sebum,
Microcomedone and comedone formation caused by hyperkeratosis of the follicular epithelium and retention keratosis,
Proliferation of microbes, particularly P. acnes in the sebum, and
Inflammation resulting from the rupture of comedones.

If not appropriately treated, acne may cause serious physical and emotional scarring and can significantly impact the quality of life of those affected by the disease. Since 1982, isotretinoin has been the treatment of choice for patients with severe nodular acne. However, its use is associated with many side effects, some of which can result in very disastrous consequences.

The ideal treatment regimen for severe acne should be efficacious but also as safe as possible. As a result, there is still an unmet medical need to improve the treatment of severe inflammatory acne vulgaris that addresses most of acne causing factors. With the invention, it is demonstrated that the efficacy/safety ratio of Adapalene-BPO associated to Doxycycline should not be inferior to that of oral Isotretinoin and provides an efficacious treatment for severe acne or nodular acne.

It is therefore the objective of this invention to provide a novel therapy regimen for the treatment of acne related diseases that avoids the adverse effects of oral isotretinoin and addresses most of acne causing factors.

### SUMMARY OF THE INVENTION

This invention provides a novel therapy regimen for the treatment of severe acne related diseases, in particular treatment of severe acne or nodular acne and/or scars. Hence, one object of the invention a therapy regimen for inhibiting or treating severe acne related diseases, comprising: (a) topically applying to an individual subject in need a therapeutically effective amount of a fixed-dose combination comprising at least one retinoid and at least one topical antibacterial agent; (b) administering a therapeutically effective amount of an oral antibiotic product with the fixed-dose combination for a predetermined period of time wherein the antibiotic is administered at a dosage ranging from 150 mg to 300 mg per day.

In particular embodiment the severe acne related diseases are selected from severe or nodular acne and/or acne scars.

The novel therapy regimen of this invention adds a topical fixed-dose combination of a retinoid and an anti-bacterial agent, such as BPO, to a course of oral antibiotic therapy. The regimen provides unexpected results for the treatment of acne related diseases. Accordingly, this invention relates to a therapy regimen for inhibiting or treating acne related diseases.

In a preferred embodiment, the duration period of treatment is 20 weeks.

The regimen includes topically applying to an individual subject in need a therapeutically effective amount of a fixed-dose combination having at least a retinoid and at least a topical antibacterial agent; administering a therapeutically effective amount of an oral antibiotic product with the fixed-dose combination for a predetermined period of time.

In a preferred embodiment, In one embodiment, the retinoid is preferably Adapalene. In an another embodiment the topical antibacterial agent is preferably benzoyl peroxide. In a preferred embodiment, the fixed-dose combination comprises Adapalene and benzoyl peroxide admixed in a pharmaceutically acceptable carrier.

According to one embodiment, the fixed-dose combination is applied once a day. In one embodiment, the fixed-dose combination is applied in the evening and the oral antibiotic product is administered in the morning.

In a preferred embodiment, the fixed-dose combination of adapalene and benzoyl peroxide is in a gel. Preferably, an aqueous gel.

According to a particular embodiment, the oral antibiotic product is selected from the group consisting of lymecycline, clindamycin, doxycycline.

Another embodiment of the inventions concerns a therapy regimen kit for inhibiting or treating severe acne related diseases comprising (a) a package containing a composition comprising at least one retinoid and at least one topical antibacterial agent; (b) a package containing an oral antibiotic product under the form of daily units comprising an antibiotic dose ranging from 150 mg to 300 mg; and (c) an instruction to facilitate patient compliance with the therapy regimen.

Another embodiment of the invention regards the use of a fixed-dose combination of a retinoid and at least an antibacterial agent for the preparation of a composition intended for inhibiting or treating severe acne-related diseases comprising: (a) topically applying to an individual subject in need a therapeutically effective amount of a fixed-dose combination comprising at least an retinoid and at least a topical antibacterial agent; (b) administering a therapeutically effective amount of oral antibiotic product with the fixed-dose combination for a predetermined period of time, wherein the antibiotic is administered at a dosage ranging from 150 mg to 300 mg per day. In a preferred embodiment, the retinoid is adapalene. In another also preferred embodiment, the antibacterial agent is the benzoyl peroxide.

The invention further concerns a fixed-dose combination comprising at least a retinoid and at least an antibacterial agent as disclosed herein, for its use for inhibiting or treating severe acne-related diseases according to a therapy regimen comprising:

(a) topically applying to an individual subject in need a therapeutically effective amount of said fixed-dose combination; (b) administering a therapeutically effective amount of oral antibiotic product with the fixed-dose combination for a predetermined period of time, wherein the antibiotic is administered at a dosage ranging from 150 mg to 300 mg per day.

Other features and advantages of this invention will be apparent from the detailed description of this invention and from the claims.

### DESCRIPTION OF FIGURES

FIG. 1 discloses the percent changes in (A) nodules, (B) papules/pustules, and (C) total lesion counts from baseline and (D) percentage of subjects with IGA success for the therapy regimen of the present invention and for a comparative therapy regimen involving oral isotretinoin, as described in the examples hereafter.

FIG. 2 discloses the mean changes in atrophic acne scar counts for the two therapy regimens above.

FIG. 3 shows the reduction of P. acnes based on % change of intensity of fluorescence for the two therapy regimens above.

### DETAILED DESCRIPTION OF THE INVENTION

We believe that one skilled in the art can, based upon the description herein, use the present invention to its fullest extent. The following specific embodiments are to be construed as merely illustrative, and do not serve to limit the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)).

### DEFINITIONS

As used herein in the specification and in the claims section below, the term “inhibit” and its derivatives refer to suppress or restrain from of occurrence or recurrence of the condition or disease to be treated, as such the regimen of this invention will reduce the likelihood for recurrence of the condition or disease to be treated.

As used herein, the term “treating” or “treatment” refers to obtaining desired pharmacological and/or physiological effect. The effect can be prophylactic or therapeutic, which includes achieving, partially or substantially, one or more of the following results: partially or totally reducing the extent of the disease, disorder or syndrome; ameliorating or improving a clinical symptom or indicator associated with the disorder; delaying, inhibiting or decreasing the likelihood of the progression of the disease, disorder or syndrome; or partially or totally delaying, inhibiting or reducing the likelihood of the onset or development of disease, disorder or syndrome.

The term “topical” and its derivatives as used herein refers to directly laying on or spreading on the skin in need of the treatment, e.g., by use of the hands or an applicator.

The term “subject” as used herein refers to mammalian animals, preferably human.

The term “therapeutically effective amount” of a therapeutic agent as used herein refers to an amount of each active component of the pharmaceutical formulation that is sufficient to show a meaningful patient benefit, i.e., to cause a decrease in, amelioration of, or prevention of the symptoms of the condition being treated. Effective amounts of the pharmaceutical formulation will vary according to factors such as the degree of susceptibility of the individual, the age, gender, and weight of the individual and idiosyncratic responses of the individual.

As used herein, the term “fixed-dose” of the therapeutic agents refers to a combination dose that is administered to a human patient without regard for the weight (WT) or body surface area (BSA) of the patient. The fixed dose is therefore not provided as a mg/kg dose or a mg/m2 dose, but rather as an absolute amount of the therapeutic agent.

As used herein, “oral” means administering a composition that is intended to be ingested. Examples of oral forms include, but are not limited to, tablets, pills, capsules, powders, granules, solutions or suspensions, and drops. Such forms may be swallowed whole or may be in chewable form. Oral forms do not include compositions intended to be topically administered to the skin.

As used herein, “pharmaceutically-acceptable” means active agents, inert ingredients, or composition that are suitable for topical or oral administration without undue toxicity, incompatibility, instability, irritation, allergic response, and the like, commensurate with a reasonable benefit/risk ratio.

### Fixed-Dose Combination

The Fixed-dose combination, according to this invention, comprises at least one retinoid and at least one topical antibacterial agent. The retinoid and the topical antibacterial agent are applied with a single topical composition comprising both the retinoid and the topical antibacterial agent.

The term “fixed dose combination” should be understood as meaning a combination whose active principles are combined at fixed doses in the same vehicle/medium (single formula) that delivers them together to the point of application. Preferably, the pharmaceutical composition in the form of a fixed combination is a gel; in this case, the two active principles are dispersed and intimately mixed, during the manufacture, in the same vehicle, which delivers them together during the application of the gel.

As used herein, the term “retinoid” refers to a class of compounds consisting of four isoprenoid units joined in a head-to-tail manner. Retinoids may be formally derived from a monocyclic parent compound containing five carbon-carbon double bonds and a functional group at the terminus of the acyclic portion. Retinoids suitable for this invention are those that are effective for treating acne. Examples of retinoids useful in this invention include tretinoin, isotretinoin, tazarotene, adapalene, benzoic acid-terminated retinoids and their heterocyclic analogs, the pharmaceutically acceptable salts and esters thereof and the like and mixtures thereof.

The term “antibacterial agent” as used herein refers to any substance of natural, semi-synthetic or synthetic origin, including all known antibiotics, that kills or inhibits the growth of one or more bacteria, but causes little or no host damage.

As used herein, the term “topical antibacterial agent” refers to a class of antibacterial agents that are suitable for topical application. Examples of such topical antibacterial agents for use herein include, but are not limited to, benzoyl peroxide (BPO), and topical antibiotics such as fluoroquinolone, β-lactam, tetracycline, macrolide, aminoglycoside, glycopeptide, linezolid, amikacin, gentamicin, tobramycin, imipenem, meropenem, cefotetan, cefoxitin, cefuroxime, cefoperazone, cefotaxime, ceftazidime, ceftozoxime, ceftriaxone, cefepime, azithromycin, ampicillin, mezlocillin, piperacillin, ticarcillin, ciprofloxacin, levofloxacin, alatrofloxacin, gatifloxacin, minocycline, chloramphenicol, clindamycin, vancomycin, cefazolin, penicillin G, nafcillin, ofloxacin, and oxacillin.

In a preferred embodiment, the topical composition comprises a therapeutically effective amount of (i) Adapalene, (ii) benzoylperoxide, and (iii) a pharmaceutically-acceptable topical carrier. The topical composition of this invention may be prepared using methodology that is well known by an artisan of ordinary skill.

Optimal dosages to be administered may be readily determined by those skilled in the art, and will vary with the particular extract(s) used, the mode of administration, the strength of the preparation, and the advancement of the disease/condition being treated. In addition, factors associated with the particular individual being treated, including individual's age, weight, diet and time of administration, will result in the need to adjust dosages.

Advantageously, the fixed-dose composition comprises between 0.0001 and 20% by weight of benzoyl peroxide (BPO) and between 0.0001 and 20% by weight of adapalene relative to the total weight of the composition; preferentially respectively between 0.025 and 10% by weight of BPO and between 0.01% and 2% by weight of adapalene relative to the total weight of the composition.

In a preferred embodiment, BPO is used with concentrations between 2% and 10% by weight and preferentially between 2.5% and 5% by weight relative to the total weight of the composition. Adapalene is used in this kind of composition in concentration between 0.01% and % by weight and preferentially between 0.01% and 0.5%, most preferred 0.1% to 0.3% by weight relative to the total weight of the composition.

A particularly preferred fixed-dose combination for use in the present invention comprises 2.5% by weight of benzoyl peroxide and 0.1% by weight of adapalene.

The fixed dose combination is a composition which may be made into a wide variety of articles that include but are not limited to ointments, lotions, creams, gels, and pastes.

Ointments, as is well known in the art of pharmaceutical formulation, are semi-solid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable-bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols of varying molecular weight; again, see Remington: The Science and Practice of Pharmacy for further information.

Lotions, are preparations to be applied to the skin surface without friction, and are typically semi-liquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Creams, as also well known in the art, are viscous liquids or semi-solid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

As will be readily be understood by those skilled in the field of pharmaceutical formulation, gels are semi-solid, suspension-type systems. Gel forming agent for use herein can be any gelling agent typically used in the pharmaceutical art for topical semi solid dosage forms. Single-phase gels contain organic macromolecules distributed substantially uniformly throughout the carrier liquid, which is typically aqueous, but also can contain an alcohol and optionally an oil. In order to prepare a uniform gel, dispersing agents such as alcohol or glycerin can be added, or the gelling agent can be dispersed by titration, mechanical mixing or stirring, or combinations thereof. The amount of gelling agents varies widely and will ordinarily range from about 0.1% to about 2.0% by weight, based on the total weight of the composition. The gel forming agent also works by the principle of copolymerization. Under alkaline pH, carbomer in presence of water undergoes cross linking and forms a gel like structure. The degree of polymerization is dependent upon the pH. At a threshold pH, the viscosities achieved by the polymer grade is the maximum.

In a specific embodiment, the said fixed-dose combination composition comprises Adapalene and Benzoyl peroxide in a form of gel such as described in WO03/055472 and incorporated herein by reference and preferably is in a form an aqueous gel.

Pastes are semi-solid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

### Antibiotic Product:

Antibiotic product suitable for the invention includes any antibiotic known by skilled in the art and appropriate to be carried out in the context of the invention. The antibiotic product for use in the invention is administrable orally.

As used herein, the term “oral antibiotic product” refers to a class of antibiotic agents that are suitable for oral administration. In a preferred embodiment, the antibiotic product is selected from the group consisting of lymecycline, clindamycin, and doxycycline. Doxycycline is preferably administered as its hyclate salt or as a hydrate, preferably monohydrate. According to the invention, a high dosage of oral antibiotic is administered, ranging from 150 mg to 300 mg, preferably from 150 to 250 mg, and more preferably at 200 mg per day.

### The Kit:

In order to facilitate compliance with the present regimen, the components thereof may be provided as a kit. The kit may include, for example, a package containing a composition comprising at least one retinoid and at least one topical antibacterial agent; (b) a package containing an oral antibiotic product under the form of daily units comprising an antibiotic dose ranging from 150 mg to 300 mg; and (c) an instruction to facilitate patient compliance with the therapy regimen in accordance with the present disclosure. The instruction for accomplishing the present regiment may be printed on the outer container of the kit or provided as a separate sheet inserted therein. It is also contemplated that the kit may optionally include a cleanser (such as, for example, a shower gel, a skin wash for the face or the body) for use in cleaning the afflicted area prior to application of the containing composition.

### The Treatment:

The therapy regimen of this invention is directed toward the treatment of severe acne related diseases, and in particular severe or nodular acne and/or acne scars. In a preferred embodiment, the acne is severe acne, preferably severe inflammatory acne vulgaris or nodular acne and/or scars.

By inflammatory acne vulgaris or nodular acne, it should be understood treatment and/or decrease in number and/or improvement of inflammatory acne lesions such as the following: Papules: A small, solid elevation less than one centimeter in diameter. Most of the lesion is above the surface of the skin.

Pustules: A small, circumscribed elevation of the skin which contains yellow-white exudates.

Nodules/Cysts: A circumscribed, elevated, lesion generally more than 1.0 cm in diameter—

Acne scars are not uniform and there are several subtypes. Some are more hypertrophic and keloidal in appearance, while others could be more atrophic (Goodman G J, et al. Postacne scarring—a quantitative global scarring grading system. Journal of Cosmetic Dermatology 2006; 5:48-52). The severity of the acne scars is correlated with the acne grade and also with the delay between the start of the disease and the start of an adapted treatment. Although not universal, acne scars generally occur with more inflammatory acne lesions that were not properly treated (Layton A M, et al. Scarred for life? Dermatology 1997; 195(suppl 1):15-21). Once established, acne scars are believed not to be treatable medically but invasive procedures could offer some improvement (Jemec G B E, Jemec B. Acne: Treatment of Scars. Clinics in Dermatology 2004; 22:434-438).

Scars are marks created during the healing of damage to the skin or tissues. A scar is permanent and cannot be completely removed.

The scars treated according to the said method, is preferentially acne scar. Specifically, acne scar is selected from atrophic scar and hypertrophic scar. More specifically acne scar is selected from ice pick, boxcar, rolling, bridges and tunnels, gross atrophy, dystrophic and keloid scars. Prio art provides definition of acne scars (Alam M, Dover J S. “Treatment of Acne Scarring.” Skin Therapy Letter. December 2006-January 2007; 11(10); Goodman G J, Baron JA. “The management of post-acne scarring.” Dermatologic Surgery. October 2007; 33(10):1175-1188.; Jacob C I, Dover J S, Kaminer M S. “Acne scarring: a classification system and review of treatment options.” Journal of the American Academy of Dermatology. 2001; 45(1): 109-117.):
Ice pick scars are deep, very narrow scars that extend into the dermis. The skin looks as if it has been pierced by an ice pick or sharp instrument. Ice pick scars seem to make a small, deep “hole” in the skin. Some may look like a large, open pore. Ice pick scars develop after an infection from a cyst or other deep inflamed blemish works its way to the surface. Skin tissue is destroyed, leaving a long column-like scar. Ice pick scars can commonly be treated with punch excision or punch grafting.
Boxar scars are depressed acne scars, round or oval in shape with steeply angled sides. They are similar in appearance to chickenpox scars. Boxcar scars occur when an inflamed acne lesion destroys tissue, leaving a sunken area on the skin. Boxcar scars may be mild and superficial, or deeper and more severe.
Rolling scars arise when fibrous bands of tissue develop between the skin and the subcutaneous tissue below. These bands pull the epidermis, binding it to deeper structures of the skin. It is this pulling of the epidermis from within that creates the rolling appearance of the skin. This type of scarring causes rolling or “wave-like” undulations across otherwise normal appearing skin. Rolling scars are best treated with subcision.
A hypertrophic scar looks like a raised, firm mass of tissue. These types of scars often grow larger than the original wound. Hypertrophic scars caused by acne are most often found on the torso, especially in men. Unlike ice pick or boxcar scars, hypertrophic scars are not caused by a loss of tissue. Rather, they develop because of an overproduction of collagen.

According to one embodiment of the invention, the therapy regimen may be administered once a day between from 12 hours to 36 hours intervals. The therapy regimen may also be administered every other day, that is, the average period of time between doses is about 48 hours, such as between 36 and 60 hours. An occasional missed dose during the course of treatment does not take the treatment regimen out of the scope of the invention.

Preferably, the fixed-dose combination composition comprising Adapalene and Benzoyl peroxide is applied in the evening and antibiotic product is administered in the morning. In a preferred embodiment, the fixed-dose combination composition comprising Adapalene and Benzoyl peroxide is applied topically and the antibiotic product is administered orally.

The duration of the therapy regimen is between 14 weeks and 25 weeks, preferably between 18 to 22 weeks and more preferably 20 weeks.

According to this invention, the topical application of the fixed dose combination composition may continue after the termination of the oral antibiotic product. The duration of such period may also been easily determined according to the labels or recommendations of the manufacturers of the therapeutic agents or products, and according to the conditions of the subjects. This enables the subjects to avoid potential bacterial resistance associated with prolonged oral antibiotic therapy.

One skilled in the art will recognize that, both in vivo and in vitro trials using suitable, known and generally accepted cell and/or animal models are predictive of the ability of an extract to treat or prevent a given condition. One skilled in the art will further recognize that human clinical trials including first-in-human, dose ranging and efficacy trials, in healthy individuals and/or those suffering from a given condition or disorder, may be completed according to methods well known in the clinical and medical arts.

### EXAMPLES

The present invention will be further illustrated below by way of Examples, but the present invention is not limited thereto.

### Example 1

### Study Protocols for Clinical Test of Treatment of Severe Acne Vulgaris with a Gel Composition Containing Adapalene 0.1%/Benzoyl Peroxide 2.5% Gel Associated with Doxycycline 200 mg Capsule Daily Versus Vehicle Gel Associated with Isotretinoin in Capsules Form

This example was the first study evaluating the concomitant use of these treatments. The purpose of this study was to show a non-inferiority in terms of efficacy/safety ratio of Adapalene 0.1%/Benzoyl Peroxide 2.5% Gel (quoted below as Adapalene BPO Gel) associated with Doxycycline Hyclate 200 mg capsules daily (quoted below as Doxycycline) compared to Adapalene 0.1%/Benzoyl Peroxide 2.5% Vehicle Gel (quoted below as Vehicle Gel) associated with Isotretinoin in the treatment of severe acne vulgaris during 20 weeks.

Isotretinoin is administrated under capsule form (of 40 mg and/or 10 mg each). The quantity administrated was calculated according to patient weight and treatment duration in order to finally administrate a dosage of 0.5 mg/kg for the first 4 weeks of treatment and 1 mg/kg for the 16 remaining weeks.

### Study Population

A total of 266 subjects were enrolled (133 in Adapalene-BPO+ Doxycycline 200 mg group and 133 in Vehicle+ Isotretinoin group)

Male or female Subjects of any race, between the age of 12 and 35 years inclusive, with a diagnosis of severe acne vulgaris and meeting specific following inclusion/exclusion criteria.

### Inclusion Criteria

1. Male or female Subjects of any race, aged 12 to 35 years inclusive,
2. Subject weighing between 50 and 110 kg
3. Subjects with severe facial acne vulgaris (global severity score at least of 4, according to Investigator's Global Assessment (IGA) scale),
4. Subjects with a minimum of 20 Inflammatory Lesions (papules and pustules) on the face, excluding the nose,
5. Subjects with a minimum of 5 nodules,
6. Female Subjects of childbearing potential with a negative urine pregnancy test at the Baseline visit and must practice a highly effective method of contraception during the study: oral/systemic [injectable, patch . . . ] contraception (must have been on a stable dose for 3 months prior to study entry), Intrauterine Device, strict abstinence, condoms, diaphragms, sponge, spermicides or partner had a vasectomy,
7. Females of non-childbearing potential, i.e., premenses, post-menopausal (absence of menstrual bleeding for 2 years), hysterectomy, bilateral tubal ligation, or bilateral ovariectomy, secondary infertility and sterility are not required to have a UPT at the beginning of the study,
8. Subjects have to read and sign the approved the Informed Consent form prior to any participation in the study. Subjects under age of majority may sign an assent form to participate in the study and they must have one parent or guardian read and sign the Informed Consent form prior to any study related procedure (but the parent or guardian is not required to attend the following visits unless requested),
9. Subjects willing and capable of cooperating to the extend and degree required by the protocol,

Study Design

This study was conducted as a multi-center, randomized, investigator-blind, controlled and parallel group trial. This non-inferiority study involved subjects of any race, aged 12 to 35 years inclusive with severe acne and meeting specific eligibility criteria.

Approximately 360 subjects were to be screened to get 300 subjects enrolled (150 in each group) in approximately 30 sites in Canada.

Subjects were randomized at baseline and treated for 20 weeks with either Adapalene-BPO gel associated with oral doxycycline or its vehicle gel associated with oral Isotretinoin.

Study Results

a. Subject Characteristics and Subject Disposition

A total of 266 subjects were enrolled across 29 centres in Canada. Of which, 217 subjects were included in the analysis-per-protocol (PP) populations (all randomized subjects without any major protocol deviations) for primary endpoint analysis. All other endpoints were analysed with population-intent-to-treat (ITT) (all randomized subjects who were dispensed study medication) (N=266).

The study population was predominantly male (85.3%), caucasian (74.8%) of phototype II or III (72.2%) with mean age of 19 years.

TABLE 1
Baseline disease characteristics (ITT)

| | Adapalene-BPO + | Vehicle + |
|---|---|---|
| | Doxy (200 mg) | Isotretinoin |
| | (N = 133) | (N = 133) |
| IGA on Face | | |
| 4: Severe | 118 (88.7%) | 115 (86.5%) |
| 5: Very Severe | 15 (11.3%) | 18 (13.5%) |
| Total lesion counts (Mean ± SD) | 104 ± 56.4 | 110 ± 64.9 |
| Nodule lesion counts (Mean ± SD) | 8 ± 3.61 | 7.56 ± 2.55 |
| Inflammatory lesion counts | 40.1 ± 22.5 | 43.4 ± 35.3 |
| (Mean ± SD) | | |
| Non-inflammatory lesion counts | 56.1 ± 46.8 | 58.6 ± 52.4 |
| (Mean ± SD) | | |
| Total atrophic acne scar counts* | 15.9 ± 16.3 | 15.5 ± 14.5 |
| (Mean ± SD) | | |

*Atrophic acne scar counts were assessed on half face

Subjects were predominantly severe (88% of IGA=4) with some very severe cases (12% of IGA=5) and presenting 107 total lesions including 8 nodules in average. Additionally, they presented 16 atrophic acne scars in average.

b. Number of Success (Efficacy/Safety)

In terms of overall success, Adapalene-BPO combined with Doxycycline (200 mg) was demonstrated to be non-inferior to isotretinoin in the PP and ITT populations. Results in the PP and ITT/Observed-case populations demonstrated not only the non-inferiority of A-BPO+doxycycline to isotretinoin but also its superiority (p=0.014 and p=0.013, respectively).

TABLE 2
Number of “success” patients efficacy/safety parts and overall (PP)

| | Adapalene-BPO + | Vehicle + |
|---|---|---|
| | Doxy (200 mg) | Isotretinoin |
| | (N = 105) | (N = 112) |
| Efficacy Success (nodule | 101 (96.2%) | 110 (98.2%) |
| reduction >75%) | | |
| Safety Success (absence of | 82 (78.1%) | 67 (59.8%) |
| medically relevant events) | | |
| Overall Success | 78 (74.3%) | 65 (58.0%) |

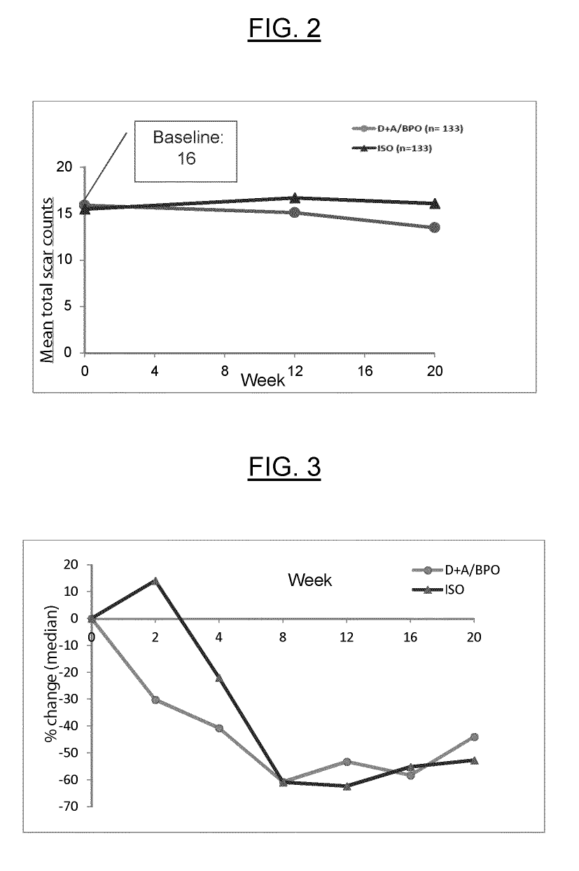

Both treatment regimens showed excellent efficacy performance (>75% reduction of acne lesions) with Isotretinoin (ISO) remaining superior to Adapalene-BPO+Doxycycline (D+A/BPO) in most of the acne lesion outcomes. Compared to isotretinoin, A-BPO+doxycycline resulted in a greater reduction at the beginning of the treatment period (statistically significant at Week 2 [p<0.01] for total acne lesions, at Week 2 and 8 for nodules [p<0.01 and p=0.011, respectively] and at Week 2 for inflammatory lesions [p<0.001]) as shown in FIG. 3.

c. Overall Success

Adapalene-BPO+Doxycycline (200 mg) regimen was demonstrated to be superior to Isotretinoin in PP and ITT population.

TABLE 3
Overall success: Primary analyses

| | | Adapalene-BPO + | Vehicle + | | | |
|---|---|---|---|---|---|---|
| | | Doxy (200 mg) | Isotretinoin | Difference | 95% CI | p-value |
| PP | N | 105 (100.0%) | 112 (100.0%) | 16.3% | [3.9%; | 0.014 |
| population | Success | 78 (74.3%) | 65 (58.0%) | | 28.6%] | |
| | Failure | 27 (25.7%) | 47 (42.0%) | | | |
| ITT | N | 116 (100.0%) | 125 (100.0%) | 14.9% | [3.1%; | 0.013 |
| Observed | Success | 85 (73.3%) | 73 (58.4%) | | 26.7%] | |
| population | Failure | 31 (26.7%) | 52 (41.6%) | | | |

Overall failures were mainly due to safety failure category that impacted nearly twice as much subjects in the Isotretinoin group than in the Adapalene-BPO+Doxycycline group (40.0% versus 23.3%).

TABLE 4
Failure Detail (ITT observed)

| | Adapalene-BPO + | | Vehicle + | |
|---|---|---|---|---|
| | Doxy (200 mg) | | Isotretinoin | |
| | (N = 116) | | (N = 125) | |
| | Event | Subject* | Event | Subject* |
| | (n) | (n, %) | (n) | (n, %) |
| AT LEAST ONE FAILURE | 47 | 31 (26.7%) | 97 | 52 (41.6%) |
| ITEM | | | | |
| Efficacy failure | 4 | 4 (3.4%) | 2 | 2 (1.6%) |
| (nodule reduction <75%) | | | | |
| Safety Failure | 43 | 27 (23.3%) | 95 | 50 (40.0%) |
| Related SAE | — | — | 1 | 1 (0.8%) |
| Related Severe AE | — | — | 5 | 5 (4.0%) |
| Related AE leading to | 5 | 4 (3.4%) | 5 | 4 (3.2%) |
| discontinuation | | | | |
| Clinically significant | 1 | 1 (0.9%) | 6 | 4 (3.2%) |
| lab abnormality | | | | |
| Related AE requiring a | 9 | 8 (6.9%) | 34 | 27 (21.6%) |
| prescription of a | | | | |
| concomitant medical | | | | |
| treatment ⁽¹⁾ | | | | |
| Predefined related AE | 1 | 1 (0.9%) | 2 | 2 (1.6%) |
| Very Bothered, at | | | | |
| least 1 Month ⁽²⁾ | | | | |
| Predefined related AE | 12 | 10 (8.6%) | 12 | 7 (5.6%) |
| Persisting at least 2 | | | | |
| Weeks ⁽³⁾ | | | | |
| Related depression | — | — | — | — |
| Predefined related AE | 9 | 7 (6.0%) | 21 | 15 (12.0%) |
| for at least 1 episode ⁽⁴⁾ | | | | |
| Inability to escalate | — | — | 5 | 5 (4.0%) |
| to Iso 1 mg/kg/day | | | | |
| Necessity of oral drug | 4 | 4 (3.4%) | 2 | 2 (1.6%) |
| downward dose | | | | |
| adjustment | | | | |
| Subject's treatment | 2 | 2 (1.7%) | 2 | 2 (1.6%) |
| concern ⁽⁵⁾ | | | | |

*A subject may have several failure items. Numbers in columns cannot be added because a given subject may have reported more than one failure item.
⁽¹⁾ AE requiring only lip balm, skin moisturizer, cleanser or other cosmetic treatment were not considered as failure. Majority of concomitant medical treatments was composed by pain killers, topical corticosteroids and antibacterial drugs.
⁽²⁾ Very Bothered, at least 1 Month: Related chelitis, xerosis, erythema, dry mouth, pruritus, epistaxis, conjunctivitis, eye irritation, dry eye.
⁽³⁾ At least 2 weeks: Related abdominal pain, vomiting, nausea, diarrhea, muscle or joint ache, headache
⁽⁴⁾ At least one episode: Related phototoxicity, allergic skin/eczematous reaction, candida vaginitis.
⁽⁵⁾ Study termination due to subject's request, providing it was motivated by treatment concern.

d. Efficacy

At Week 20, Adapalene-BPO+Doxycycline showed excellent efficacy performance with more than 75% median reduction on all acne lesions. However, Isotretinoin regimen remained superior to Adapalene-BPO+Doxycycline in most of the acne lesion outcomes.

TABLE 5
Lesions % change at Week 20 (ITT LOCF)

| | Adapalene-BPO + | Vehicle + | |
|---|---|---|---|
| | Doxy (200 mg) | Isotretinoin | |
| | (n = 133) | (n = 133) | p-value |
| Total lesion counts | −78 | −93 | <.001 |
| (Median) | | | |
| Inflammatory lesions | −80 | −95 | <.001 |
| (Median) | | | |
| (not including nodules) | | | |
| Nodules (Mean) | −89 | −96 | <.001 |

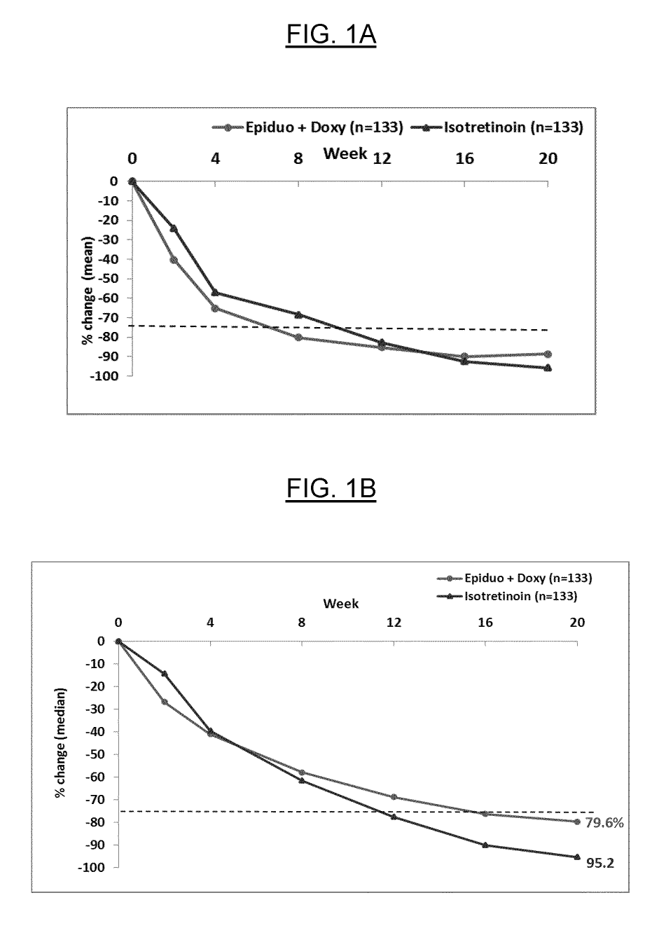

Lesions changes outcomes are presented in FIG. 1 (A) nodule, (B) papules/pustules, and (C) total lesion counts from baseline and (D) percentage of subjects with IGA success.

No increase of atrophic acne scars was observed in both groups throughout the 20-week treatment period, suggesting a comparable effect of both regimens on prevention of atrophic acne scars appearance.

TABLE 6
Atrophic acne scar: Count at each
evaluation visit, Descriptive (ITT)

| | Adapalene-BPO + | Vehicle + |
|---|---|---|
| | Doxy (200 mg) | Isotretinoin |
| | (n = 133) | (n = 133) |
| Baseline (ITT) (Mean ± SD) | 15.9 ± 16.3 | 15.5 ± 14.5 |
| W12-LOCF(ITT) (Mean ± SD) | 15.1 ± 14.8 | 16.7 ± 15.7 |
| W20 -LOCF (ITT) (Mean ± SD) | 13.5 ± 12.8 | 16.1 ± 16.7 |

e. Safety

Adapalene-BPO with Doxycycline regimen showed a much safer profile than Isotretinoin.

Substantially fewer subjects reported at least 1 related AE with Adapalene-BPO+Doxycycline regimen compared to the Isotretinoin regimen (52.6% vs 88%).

In a total of 441 treatment-related AE, 299 AE resulted from the Isotretinoin group and 142 from the Adapalene-BPO+Doxycycline group.

Out of these 441 treatment-related AE, 106 were deemed as a safety failure; of which, 73 resulted from the Isotretinoin group and 33 from the Adapalene-BPO+Doxycycline group. These AEs of special management concern impacted nearly twice as much subjects in the Isotretinoin group compared to Adapalene-BPO+Doxycycline group (33.8% vs 18%).

TABLE 7
Overview of related adverse events (APT)

| | Adapalene- | | | | | |
|---|---|---|---|---|---|---|
| | BPO + | | Vehicle + | | | |
| | Doxy (200 mg) | | Isotretinoin | | Total | |
| | (N = 133) | | (N = 133) | | (N = 266) | |
| | | Subj.* | | Subj.* | | Subj.* |
| | Evt n | n (%) | Evt n | n (%) | Evt n | n (%) |
| Related AEs | 142 | 70 (52.6%) | 299 | 117 (88.0%) | 441 | 187 (70.3%) |
| Related AEs deemed as | 33 | 24 (18%) | 73 | 45 (33.8%) | 106 | 69 (25.9%) |
| safety failure⁽¹⁾ | | | | | | |
| Related serious AEs | 0 | 0 | 1 | 1 (0.8%) | 1 | 1 (0.4%) |
| Related severe AEs | 0 | 0 | 5 | 5 (3.8%) | 5 | 5 (1.9%) |
| Related AEs leading to | 5 | 4 (3.0%) | 5 | 4 (3.0%) | 10 | 8 (3.0%) |
| discontinuation | | | | | | |
| Related AE requiring a | 9 | 8 (6.0%) | 34 | 27 (25.6%) | 43 | 32 (12.0%) |
| prescription of a | | | | | | |
| concomitant medical | | | | | | |
| treatment | | | | | | |
| Clinically significant | 1 | 1 (0.8%) | 6 | 4 (3.0%) | 7 | 5 (1.9%) |
| lab abnormality | | | | | | |
| Other medically | 22 | 17 (12.8%) | 35 | 23 (17.3%) | 57 | 40 (15.0%) |
| relevant related AEs⁽²⁾ | | | | | | |

*Subjects with at least one event. Numbers in columns cannot be added because a given subject may have reported more than one AE.
⁽¹⁾Each AE is counted once whatever the number of safety failure criteria met by the AE.
⁽²⁾Gathering following related AE safety failure categories: Very Bothered, at least 1 Month + Persisting at least 2 Weeks + At least 1 episode.

Additionally, it can be noticed that no related SAE or severe AE reported in the Adapalene-BPO+Doxycycline group, while 1 related Serious AE (erythema multiforme major requiring hospitalization) and 5 related severe AE (i.e. severe dry lips, severe erythema multiforme major, severe fatigue and severe acne flare) reported in the Isotretinoin group;

Similar number of related AE leading to discontinuation (i.e. 5) was reported in both groups

### DEFINITIONS

### Adverse Events (AE)

An adverse event (AE) can be any unfavorable and/or unintended sign (including an abnormal laboratory finding), symptom, or disease temporally associated with the use of a medicinal/investigational product, whether or not related to the medicinal/investigational products or to the study procedures.

Thus any new sign, symptom or disease, or clinically significant increase in the intensity of an existing sign, symptom or disease, were considered as an adverse event.

### Notes:

Clinically significant worsening of the disease/condition being evaluated, which occurs during the study, was considered an adverse event,

Any new sign or symptoms suffered by the Subject which appeared after accidental or intentional overdose or misuse were also reported as an adverse event.

Any adverse event, whether or not it is related to the investigational products or to the study procedures, was reported on the Adverse Event form along with the diagnosis preferably or signs/symptoms description, the date of onset, the severity, the seriousness, the relationship and the action taken with the investigational product but also the treatment given to treat the AE and the final AE outcome.

Assessment of AE seriousness, severity and causality was based on specific definitions.

If the Subject discontinued due to an Adverse Event, the Adverse Event and Exit Forms had to be completed.

Side effects may be expected during topical study treatment, the characteristics of which are described in this protocol (e.g., erythema, scaling, dryness, and stinging/burning). The course of these expected events was assessed and reported on the tolerability assessments. An Adverse Event Form was completed only if the severity of the expected signs and symptoms was such that an interruption of the Subject's participation in the study occurred at Investigator's decision and/or if a concomitant medication (except provided moisturizers) was prescribed to treat the sign/symptom.

### Serious Adverse Events (SAE)

A Serious Adverse Event is any untoward medical occurrence that at any dose:
results in death,
is life-threatening,
requires inpatient hospitalization or prolongation of existing hospitalization,
results in persistent or significant disability/incapacity, or
is a congenital anomaly/birth defect.
   And also:
Other important medical events that jeopardize the Subject or require intervention to prevent one of the outcomes listed above.

### Note:

The term “life-threatening” refers to an event in which the Subject was at risk of death at the time of event; it does not refer to an event which hypothetically might have caused death if it was more severe.
Hospitalization solely for the purpose of diagnostic tests, even if related to an adverse event, elective hospitalization for an intervention which was already planned before the inclusion of the Subject in the study, and admission to a day-care facility may not themselves constitute sufficient grounds to be considered as a serious adverse event.

Relationship to Study Drugs

The relationship assessment for an adverse event was completed using the following definitions as a guideline for all adverse events occurring during clinical trials conducted or sponsored by Galderma:

| | |
|---|---|
| Definitely | Should be reserved for those events which occur prior to |
| unrelated: | investigational product(s) administration (e.g., washout or |
| | single-blind placebo) or for those events which cannot be |
| | even remotely related to study participation (e.g., injuries |
| | sustained as a passenger in an automobile accident). |
| Unlikely: | There is no reasonable temporal association between the |
| | investigational product(s) and the event and the event could |
| | have been produced by the Subject's clinical state or other |
| | modes of therapy administered to the Subject. |
| Possible: | The event may or may not follow a reasonable temporal |
| | sequence from investigational product(s) administration but |
| | seems to be the type of reaction that cannot be dismissed as |
| | unlikely. The event could have been produced or mimicked by |
| | the Subject's clinical state or by other modes of therapy |
| | concomitantly administered to the Subject. |
| Probable: | The event follows a reasonable temporal sequence from |
| | investigational product(s) administration, abates upon |
| | discontinuation of the investigation product, and cannot be |
| | reasonably explained by the known characteristics of the |
| | Subject's clinical state. |
| Definitely | Should be reserved for those events which have no uncertainty |
| related: | in their relationship to investigational product(s) |
| | administration: this means that a re-challenge was positive. |

For AEs graded possible, probable or definitely related; the Investigator determined whether is related to topical or oral investigational product or both.

The following populations was analyzed:

### The Intent-to-Treat Efficacy Population (ITT)

This population consisted of the entire population enrolled and randomized (i.e., assigned a treatment (or kit) number.

### The Safety Population (APT)

This population consisted of the intent-to-treat population, after exclusion of Subjects who never took the treatment with certainty based on monitoring report.

### Missing Values

For efficacy variables, in order to evaluate the effect of major deviations or of data exclusions, the ITT population were analyzed at each visit using the last observation carried forward (LOCF) to impute missing values. If no post-baseline data was available, baseline was carried forward. Thus, the number of Subjects did not vary at each visit. The other missing values were not replaced (observed data).

### Data Presentation and Graphics

All continuous data were summarized using usual statistics: number of values, mean, median, standard deviation, minimum and maximum, and by frequency distribution (n, %) for qualitative data. For ordinal data, both frequency distribution and usual statistics were presented. All tables were presented by study medication and by visit.

For the safety variables, all summaries were based on the safety population (APT). The adverse events were descriptively summarized (n, %) by relationship to investigational products and by intensity (i.e. mild, moderate and severe). In the case of an adverse event occurring more than once during the study period, the adverse event with the highest drug related rating in that period was used in the summary by categories of relationship to drug. Similarly, the adverse event with the highest intensity in that period was used in the summary by intensity. The adverse experiences were summarized (n, %) within SOC and preferred term (MedDRA). Deaths and serious adverse events were reported as well as withdrawals due to adverse events.

The objective of this study was to demonstrate the non-inferiority in terms of efficacy/safety ratio of Adapalene-BPO gel associated with 200 mg Doxycycline capsules compared to vehicle gel associated with Isotretinoin capsules.

To achieve this objective, a primary outcome, overall success, was defined as a composite endpoint including efficacy and safety measurements.

Overall success was deemed to be reached when the 2 following criteria were fulfilled:

### 1—Efficacy:

Reduction of at least 75% of number of nodules at the end of treatment

### 2—Safety:

### Absence of any Safety Failures Listed Below:

Any related serious adverse event (SAE)
Any related severe adverse event (AE)
Any related AE leading to discontinuation of treatment
Any related AE requiring a prescription of a concomitant medical treatment
Inability to escalate to 1 mg/kg/day at week 4 for isotretinoin
Necessity of downward treatment adjustment (for both oral treatments)
Study termination due to subject's request, providing it is motivated by treatment concern
If not already covered by the above list, other specific safety failures as listed below:
   Related chelitis, xerosis, erythema, dry mouth, pruritus, epistaxis, conjunctivitis, eye irritation, dry eye, if the subject is “very bothered” for at least one month (3-point scale: “not bothered”, “bothered”, “very bothered”)
   Related abdominal pain, vomiting, nausea, diarrhea, muscle or joint ache, headache, if persisting at least 2 weeks
   Related depression (moderate to severe depression according to ICD-10/MDI)
   Related phototoxicity, allergic skin/eczematous reaction, candida vaginitis, for at least one episode
   Related biological abnormality clinically significant

The non-inferiority would be demonstrated by showing that the 95% confidence interval of the between treatment difference excludes a 15% inferiority in terms of overall success, on PP and ITT population.

Other secondary efficacy endpoints were the percent change from baseline visit in Total lesion counts, Inflammatory lesion counts, Nodule counts, and the total atrophic acne scar counts.

Safety was assessed by recording of adverse events.

### Example 2

### Product Formulations for the Study of Example 1

### 1. Epiduo Gel

Epiduo gel containing Adapalene 0.1%/Benzoyl Peroxide 2.5% Gel was used for the study. Each subject applied Epiduo gel once daily in the evening.

Epiduo gel has the following formulation (expressed as % weight/total weight).

| | |
|---|---|
| Adapalene | 0.10% |
| Benzoyl peroxide | 2.50% |
| Copolymer of acrylamide & sodium | 4.00% |
| acryloyldimethyltaurate | |
| Sodium docusate | 0.05% |
| Disodium EDTA | 0.10% |
| Glycerol | 4.00% |
| Poloxamer 124 | 0.20% |
| Propylene glycol | 4.00% |
| Purified water | qs 100% |

### 2. Doxycycline Hyclate

Doxycycline Hyclate 200 mg capsule was used for the study. Subject selection and dosage for Doxycycline were based on the USA/Canada approved labeling of Doxycycline. Each subject toke Doxycycline once daily in the morning with a meal.

### 3. Vehicle Gel

The result with the vehicle gel, which is essentially the same formulation of Epiduo Gel without adapalene and benzoyl peroxide was used as baseline for the study.

### Conclusions of Examples 1 and 2

In terms of overall success, Adapalene-BPO combined with Doxycycline (200 mg) was demonstrated to be non-inferior to Isotretinoin in the PP population. This result was confirmed in the ITT analysis. Results in the PP and ITT/Observed-case populations demonstrated not only the non-inferiority of A-BPO+doxycycline to isotretinoin but also its superiority (p=0.014 and p=0.013, respectively).

Both treatment regimens showed excellent efficacy performance (>75% reduction of acne lesions) Additionally, no increase of atrophic acne scars was observed in both groups throughout the 20-week treatment period, suggesting a comparable effect of both regimens on prevention of atrophic acne scars appearance.

As for safety, Adapalene-BPO with Doxycycline showed a much safer profile than Isotretinoin: in a total of 441 treatment-related AE, 299 AE resulted from the Isotretinoin group and 142 from the Adapalene-BPO+Doxycycline group. Out of these 441 treatment-related AE, 106 were deemed as a safety failure; of which, 73 resulted from the Isotretinoin group and 33 from the Adapalene-BPO+Doxycycline group. These AEs of special management concern impacted nearly twice as much subjects in the Isotretinoin group compared to Adapalene-BPO+Doxycycline group (33.8% vs 18%).

A composite endpoint including both efficacy and safety criteria showed that Adapalene-BPO+Doxycycline 200 mg was non-inferior to Isotretinoin, as demonstrated in the per-protocol (74.3% vs. 58%; p=0.014) and intent-to-treat (63.9% vs. 54.9%; p=0.125) populations.

Adapalene-BPO+Doxycycline 200 mg has a favorable benefit/risk profile compared to Isotretinoin in treatment of severe nodular acne and demonstrate an earlier onset of effect in inflammatory and total lesions.

These findings suggest that Adapalene-BPO combined with Doxycycline (200 mg) is an interesting alternative to oral Isotretinoin for effective and safe treatment of severe nodular acne patients.

Atrophic acne scar counts were conducted on half the face and the presence of P. acnes were also assessed by UV fluorescence photography (Visia® system—Canfield Scientific, Inc.). The presence of P. acnes has been shown to correlate with intensity of orange red fluorescence from its metabolites (coproporphyrin III).¹⁶ This method measures the total spot area, or number of pixels associated with UV fluorescent spots, and when reduced indicates a decrease in the presence of P. acnes. Digital fluorescence photography has been found to be reliable, quick and practical, and porphyrin fluorescence correlated well with the decrease in P. acnes density from scrub cultures.¹⁷ Though it is not sensitive enough to detect slight changes in P. acnes, it is highly reliable when large variations occur, indicating clinical relevance. As few centers were equipped with this system, this test was performed on a subset of only 40 subjects.

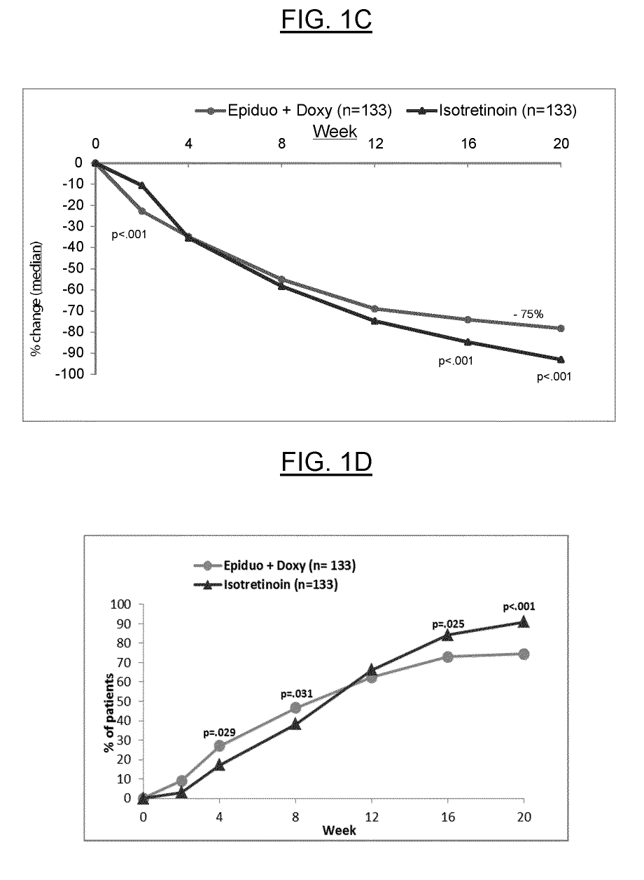

The mean number of total atrophic acne scars assessed at baseline for each half of the face was about 16 in each group (FIG. 2). After 20 weeks of treatment, no change in acne scar counts was observed in either group.

Similarly to lesions and IGA results, starting at week 2, an earlier onset of action in favor of Adapalene/BPO+Doxycyclin was observed in the reduction of P. acnes fluorescence (FIG. 3), with a median percent change of—30.2% vs. +14.1%, respectively. At week 20, P. acnes reduction was in favor of ISO.

Adapalene/BPO+Doxycyclin demonstrates a comparable, non-inferior benefit/risk to Isotretinoin over 20 weeks in the treatment of severe nodular acne, and may be an alternative option for those unwilling or unable to use the latter due to contraindications. As Adapalene/BPO+Doxycyclin is more efficacious in reduction of papules/pustules, nodules and total acne lesions at 2 weeks, this combination may be particularly indicated where rapid efficacy is desirable.

1. A therapy regimen for inhibiting or treating severe acne related diseases, the regimen comprising: (a) topically applying to an individual subject in need thereof a therapeutically effective amount of a fixed-dose combination comprising at least one retinoid and at least one topical antibacterial agent; (b) administering a therapeutically effective amount of an oral antibiotic with the fixed-dose combination for a predetermined period of time, wherein the antibiotic is administered at a dosage ranging from 150 mg to 300 mg per day.

2. The regimen according to claim 1, wherein the severe acne related diseases are selected from the group consisting of severe inflammatory acne vulgaris, nodular acne and acne scars.

3. The regimen according to claim 1, wherein the regimen decreases the number of inflammatory acne lesions selected from the group consisting of papules, pustules, nodules and cysts.

4. The regimen according to claim 1, wherein the duration of treatment is 2 weeks.

5. The regimen according to claim 1, wherein the duration of step b) ranges from 14 weeks to 25 weeks.

6. The regimen according to claim 1, wherein the retinoid is adapalene.

7. The regimen according to claim 1, wherein the topical antibacterial agent is benzoyl peroxide.

8. The regimen according to claim 1, wherein the fixed-dose combination is a composition comprising adapalene and benzoyl peroxide admixed in a pharmaceutically acceptable carrier.

9. The regimen according to claim 1, wherein the fixed-dose combination is applied once a day.

10. The regimen according to claim 8, wherein the composition is a gel.

11. The regimen according to claim 10, wherein the composition is an aqueous gel.

12. The regimen according to claim 8, wherein the composition comprises from 0.0001% to 20% by weight of benzoyl peroxide and from 0.0001% to 20% by weight of adapalene relative to the total weight of the composition.

13. The regimen according to claim 12, wherein the composition comprises from 2% to 10% by weight of benzoyl peroxide.

14. The regimen according to claim 12, wherein the composition comprises from 0.01% to 1% by weight of adapalene relative to the total weight of the composition.

15. The regimen according to claim 1, wherein the oral antibiotic is selected from the group consisting of lymecycline, clindamycin, and doxycycline

16. The regimen according to claim 15, wherein the oral antibiotic is doxycycline.

17. The regimen according to claim 1, wherein the oral antibiotic is in the form of a capsule.

18. The regimen according to claim 1, wherein the oral antibiotic is administered at a daily concentration ranging from 150 mg to 250 mg.

19. A method of preparing a composition to inhibit or treat severe acne-related diseases, the method comprising:
(a) formulating the composition to be topically applied to an individual subject in need: (b) preparing the composition to comprise a therapeutically effective amount of a fixed-dose combination comprising at least one retinoid and at least one antibacterial agent; and (c) formulating the composition to be effectively administered with a therapeutically effective amount of an oral antibiotic for a predetermined period of time, wherein the antibiotic will be administered at a dosage ranging from 150 mg to 300 mg per day.

20. The method according to claim 19, wherein the retinoid is adapalene.

21. The method according to claim 19, wherein the antibacterial agent is benzoyl peroxide.

22. A therapy regimen kit for treating severe acne related diseases, the kit comprising:
(a) a composition comprising at least one retinoid and at least one topical antibacterial agent;
(b) an oral antibiotic that can be administered in an antibiotic dose ranging from 150 mg to 300 mg daily; and
(c) an instruction to facilitate patient compliance with the therapy regimen.

23. A fixed-dose combination comprising at least one retinoid and at least one antibacterial agent as defined in claim 1, wherein the fixed-dose combination is effective in inhibiting or treating severe acne-related diseases according to a therapy regimen wherein:
(a) the fixed-dosed combination is topically applied to an individual subject in need thereof; and (b) a therapeutically effective amount of oral antibiotic is administered with the fixed-dose combination for a predetermined period of time, wherein the antibiotic is administered at a dosage ranging from 150 mg to 300 mg per day.

24. The fixed-dose combination of claim 23, wherein the therapy regimen is as defined in claim 2.

25. The regimen according to claim 5, wherein the duration of step b) is from 18 weeks to 22 weeks.

26. The regimen according claim 25, wherein the duration of step b) is 20 weeks.

27. The regimen according to claim 12, wherein the composition comprises from 0.025% to 10% by weight of benzoyl peroxide and from 0.01% and 2% by weight of adapalene relative to the total weight of the composition.

28. The regimen according to claim 13, wherein the composition comprises 2.5% to 5% by weight of benzoyl peroxide relative to the total weight of the composition.

29. The regimen according to claim 14, wherein the composition comprises from 0.01% to 0.5% by weight of adapalene relative to the total weight of the composition.

30. The regimen according to claim 29, wherein the adapalene is present in the composition in an amount of from 0.1% to 0.3% by weight relative to the total weight of the composition.

31. The regimen according to claim 18, wherein the oral antibiotic is administered at a daily concentration of 200 mg.
